(19) United States
(12) Patent Application Publication
(10) Pub. No.: US 2004/0219229 A1
(43) Pub. Date: Nov. 4, 2004

(54) Migraine relief composition and methods of using and forming same

(76) Inventor: Tim Clarot, Phoenix, AZ (US)

Correspondence Address: SNELL & WILMER, ONE ARIZONA CENTER, 400 EAST VAN BUREN, PHOENIX, AZ, 850040001

(21) Appl. No.: 10/427,352

(22) Filed: Apr. 30, 2003

(51) Int. Cl. A61K 35/78; A61K 33/06; A61K 31/525

(52) U.S. Cl. 424/682; 424/764; 514/251

(57) ABSTRACT

A composition to treat symptoms of migraines and/or prevent the onset of migraines and methods of forming and using the composition are disclosed. The composition is formulated to maintain one or more active ingredients in association with a nasal and/or oral membrane for an extended period of time. The invention further includes a system and method for applying the gelled composition to the nasal membrane.

### FIELD OF INVENTION

The present invention relates generally to a composition for reducing symptoms associated with a migraine. More particularly, the invention relates to a viscous composition that includes one or more active ingredients for treating migraines and to methods of using and forming the composition.

### BACKGROUND OF THE INVENTION

Millions of people each year suffer from migraine symptoms. Exemplary migraine symptoms include one or more of the following: intense throbbing headache, often on only one side of the head; nausea; vomiting; diarrhea; increased sensitivity to light; increased sensitivity to sounds; and increased sensitivity to smells. Because the symptoms can be quite severe and even debilitating, it is desirable to have fast-acting relief to apply at the early onset of such symptoms and/or compositions that prevent the onset of the symptoms.

Typical migraine relief remedies are generally administered orally. Although such remedies may work well for relieving some migraine symptoms, the effectiveness of many of these remedies may be limited due to, for example, digestive processes in the oral and digestive pathways. For example, enzymatic activity in the oral cavity and/or acidic environments present in the digestive system may degrade the performance of specific elements or compounds comprising active substances in migraine relief compositions. A further disadvantage of typical compositions involves the circuitous routes some active ingredients are forced to travel, such as when orally administered substances must travel from the oral cavity though the digestive system before being absorbed though portions of the digestive system.

Accordingly, a fast-acting composition for treating migraine symptoms and/or preventing the onset of the symptoms and methods of using and forming the compositing are desired.

### SUMMARY OF THE INVENTION

The present invention provides improved methods and compositions for treating migraines. While the ways in which the present invention addresses the deficiencies and disadvantages of the prior art are described in greater detail hereinbelow, in general, according to various aspects of the present invention, a method and composition are provided for quickly delivering relief to migraine sufferers.

In accordance with various embodiments of the present invention, a migraine composition is formulated to permit the active substance to remain in contact with an oral or nasal membrane for an extended period of time. Maintaining the active ingredients in contact with the nasal and/or oral membrane for an extended period of time facilitates absorption of the active ingredient through the membrane, which in turn increases the speed of symptom relief.

In accordance with one exemplary embodiment of the invention, a composition for application to a nasal membrane is configured to maintain an active substance, such as Magnesia Sulphurica (magnesium sulfate: MgSO₄), Magnesia Muriatica (magnesium chloride: MgCl₂), Riboflavinum (Riboflavin), Chrysanthemum Leucanthemum, or any combination thereof in contact with the nasal membrane for an amount of time sufficient to permit delivery of a desired amount of the active substance to active sites on the nasal membrane, and in further embodiments, across the nasal membrane into the blood or circulatory system of a patient. In accordance with various aspects of this embodiment, the active ingredients include a homeopathic agent. In accordance with further aspects, the active ingredients include an allopathic agent. In accordance with various other aspects of this embodiment, the composition includes a gelled delivery matrix, the viscosity of which is formulated to promote extended contact of the active ingredient(s) with the nasal membrane while still allowing efficient migration of the active substance(s) across the gelled matrix for delivery to the nasal membrane.

In accordance with additional embodiments of the invention, the composition includes a homeopathic amount of a calming agent, an anti-spasmodic agent, a serotonin antagonist, a vessel tone agent, or any combination thereof.

In accordance with various other embodiments of the invention, a system for delivering the composition to the nasal membrane is provided. In accordance with aspects of this embodiment, a spray applicator is provided for applying the composition to the nasal membrane.

In accordance with another embodiment of the invention, a composition for treating a migraine is formed by admixing a solvent, one or more active ingredients, a carrier, and a thickening agent. The composition is mixed until a desired consistency is obtained.

In accordance with yet another embodiment of the invention, a method of using the migraine composition includes applying a desired amount of the composition to a nasal membrane. In accordance with various aspects of this embodiment, a metered amount of the composition is applied to the nasal membrane using a spray pump.

### DETAILED DESCRIPTION

In accordance with various aspects of the present invention, a composition is provided, which includes a gelled matrix composition and one or more active substances. The composition is formulated to promote maintenance of the active substance in direct contact with at least a portion of the nasal or oral cavity for an extended period of time.

In accordance with exemplary embodiments of the invention, a migraine relief composition includes one or more active ingredients and a carrier. As described below, the composition may also include additional ingredients such as buffers, stabilizers, and preservatives.

As used herein, an active substance includes any of one or more substances that produces or promotes a beneficial therapeutic, physiological, homeopathic, allopathic and/or pharmalogical effect on the body. Such beneficial effects may be brought upon any animal or human patient, and various systems associated therewith, including the immune system, respiratory system, circulatory system, nervous system, digestive system, urinary system, endocrine system, muscular system, skeletal system, and the like, as well as any organs, tissues, membranes, cells, and subcellular components associated therewith.

As will be appreciated by those skilled in the art, beneficial effects include reducing pain associated with a migraine (e.g., as measured using the classical 4-point pain severity index scale); reduction in duration of a migraine; reduction in occurrence of migraines; reduction of light, sound, and/or smell sensitivity; and/or reduction or elimination of diarrhea, vomiting, and/or nausea.

Exemplary active substances include calming agents such as Chrysanthemum Leucanthemum (e.g., prepared in 45% by weight alcohol), anti vessel swelling agents such as Magnesia Sulphurica and Magnesia Muriatica, and serotonin antagonists such as Riboflavinum, as well as various combinations of these agents.

The carrier is configured to maintain the active ingredient in contact with an oral or nasal membrane and to facilitate transport of the active ingredient from the bulk composition to the membrane. In accordance with various embodiments of the invention, the carrier includes at least one fluid component and one thickener component. Exemplary fluid components include any suitable fluid or liquid, such as, for example, water, oil, alcohol, etc. Likewise, the thickener component may include any acceptable thickener (e.g., a substance which increases the viscosity of the composition, causes the composition to gel or coagulate, or the like), such as food-grade or pharmaceutical grade thickeners, including, for example, glycerin, carrageenan, sugar, guar gum, methylcellulose, aloe vera, hydroxyethyl cellulose, and the like. The carrier may also include other gels, gelling agents, antiseptics, preservatives, permeation enhancers, sequestering agents, buffers, emulsifiers, or similar ingredients.

In accordance with various embodiments of the present invention, the composition is a homeopathic or allopathic composition, which includes from about 75% to about 99.999% by weight of at least one carrier and an effective amount of an active substance. The effective amount of the active substance includes any amount of active substance required in a composition or dose suitable to render a beneficial therapeutic effect. For example, in accordance with one aspect of this embodiment, the composition includes from about 0.0000001% to about 10% by weight of at least one active substance.

In accordance with various other embodiments, the composition is a pharmaceutical composition, which includes 75% to 99.999% by weight of at least one carrier and an effective amount of an active substance. The effective amount of the active substance includes from about 0.0000001% to about 10% by weight of the composition of at least one active substance.

Exemplary compositions of the present invention have a viscosity from about 2,500 to about 40,000 centipoise, and preferably from about 3,000 to about 10,000 centipoise, and more preferably from about 5,000 to about 9,000 centipoise. In accordance with one aspect of the invention, the composition is formulated to maintain the settled matrix in association with the nasal cavity for an extended duration. Preferably, this extended duration provides an amount of time sufficient to permit delivery of the active substance to the nasal membrane. When the viscosity is less than about 2,500 centipoise, the composition tends to be drawn out of the nasal cavity by gravity. Compositions exceeding 40,000 centipoise tend to impede transport and delivery of the active substance to the nasal membrane. In some embodiments, the formulation may facilitate maintenance of the matrix in the nasal cavity for about at least ten minutes. In still other embodiments, the formulation may permit maintenance of the matrix in the nasal cavity for about 12 hours. The viscosity noted above was measured on compositions at about 25° C. using a Brookfield viscometer set at about 3 rpm with spindle T-A. The viscosity measurements may be made prior to or after administration of the composition, such as after application from a nasal spray applicator to a selected surface. In accordance with various embodiments of the invention, the composition may thicken after being forced through the spray device, or where the composition is admixed with other components.

The viscous formulation impedes downward flow of the composition out of the nasal cavity due to various counteracting forces, which among others include gravitational forces. The composition of the present invention provides a gelled composition of sufficient viscosity to impede the downward flow of the applied solution out of the nasal cavity, thereby facilitating the delivery of an effective amount of active ingredient to the nasal membrane.

In accordance with a further aspect of the present invention, the composition provides an efficient distribution and transport system for delivery of the active substance through the gelled composition to the nasal membrane. In accordance with the present invention, after application of the gelled composition, an interface layer is thought to form between the gelled composition and the nasal membrane. As the active substance is delivered to the nasal membrane, the concentration of the active substance at the interface layer becomes depleted. Due to the unique properties of the gel, additional amounts of the active substance are permitted to travel down the resulting concentration gradient, from higher concentration to lower concentration, to replenish the concentration of the active substance at the interface layer, thereby further driving additional amounts of the active substance into contact with the nasal membrane. This transport system may be impeded however where the viscosity of the gelled composition is too high, such as, for example, where it exceeds 40,000 centipoise.

The gelled composition preferably permits the active substance to diffuse through the composition to the nasal epithelial membrane or mucous of the epithelial membrane. This facilitates the availability of a regular supply of the active substance because diffusion within the composition continues to supply the active substance without requiring that the portion of the composition adjacent to the nasal epithelial membrane (or mucous on the membrane) dissolve or dissipate and expose a fresh portion of the composition containing the active substance. As noted, composition viscosities in excess of about 40,000 centipoise are believed to interfere with the diffusion of active substances through the composition.

In a preferred embodiment of the present invention, the gelled matrix is formed from a carrier composition including from about 0.05% to about 5.0%, and preferably about 1.0 by weight glycerin. Glycerin is thought to allow ionic active ingredients to remain in an ionic state until contact with the nasal membrane and/or mucous of the nasal membrane. Glycerin is also preferred because it has the ability to permeate nasal mucous and the nasal epithelial membrane, while carrying with it the active substance for appropriate delivery.

As noted above, the composition may also include permeation enhancers, which are believed to function by enlarging or loosening tight junctions between cells in the nasal membrane, thereby facilitating passage of the active substance therethrough. Exemplary permeation enhancers include liposomes, sequestering agents, ascorbic acid (Vitamin C), glycerol, chitosan, and lysophosphotidylcholin, or any other substance that provides a similar function or result. By way of example, the permeation enhancer may include a sequestering agent, such as EDTA. EDTA is thought to chelate calcium. When applied to the nasal membrane, it is believed to remove calcium from the cell junctions, thereby loosening the junctions to facilitate passage of an active substance therethrough.

Permeation enhancers may be present in any effective amount, with preferably concentrations ranging from about 0.00001% to about 5.0% by weight. In a preferred embodiment, the permeation enhancer includes disodium EDTA, at a concentration of about 0.0001% to about 1.0% by weight, and preferably at about 0.10% by weight.

In accordance with another aspect of the invention, a preservative may be added to the composition to facilitate stability of the various ingredients. Any suitable preservative may be used in accordance with the present invention. For example, the preservative may include an alcohol—e.g., benzyl alcohol at a concentration of about 0.0001% to about 1.0% by weight, and preferably 0.2% by weight. Preferably, the preservative includes a 50% solution of benzalkonium chloride, admixed into the composition at a concentration of about 0.0001% to about 0.1% by weight, and preferably about 0.02% by weight. Benzalkonium chloride is another suitable preservative or antiseptic. The preservative may also comprise from about 0.001% to about 1.0% by weight and preferably about 0.10% by weight disodium EDTA. It is believed disodium EDTA facilitates stability by combining with metals (chelating) and further by preventing various oxidative processes.

The composition may also include at least one buffer. Any suitable buffer may be used in accordance with the present invention. In an exemplary embodiment, the composition includes from about 0.0001% to about 1.0%, and preferably about 0.11% by weight of sodium phosphate; about 0.0001% to about 3.0%, and preferably about 0.02% by weight of potassium chloride; about 0.0001% to about 3.0%, and preferably about 0.012% by weight of potassium phosphate; about 0.0001% to about 3.0%, and preferably about 0.9% by weight of sodium chloride as perservatives.

In accordance with various aspects of the invention, viscosity may be determined prior to or after application of the composition. For example, it may be desirable to utilize a nasal gel composition which thickens when placed in the nostril of a patient. Utilizing a component which is temperature sensitive and thickens due to the increased temperature in a patient's nose is one avenue of producing an increased viscosity when the nasal gel is applied in the patient's nose. Another avenue is to admix two or more components just prior to applying the nasal gel in the patient's nose. In this case, the two components produce a composition having a viscosity greater than either component separately.

The composition of the present invention may be delivered to the nose cavity according to any suitable method, such as a drop applicator, cotton swab, etc. In accordance with a preferred embodiment of the present invention, the composition is delivered as a spray. The method includes the steps of obtaining a composition in accordance with the present invention for delivery into the nasal cavity. The method further includes the step of applying the delivery composition in the nasal cavity with a spray applicator. In further embodiments, the method also includes the step of applying the delivery composition to the nasal cavity such that a first portion of the composition directly contacts at least the nasal membrane, a second portion of the composition directly contacts at least the mucous in the nasal cavity, and a third portion of the composition directly contacts at least the cilia in the nasal cavity.

Practitioners will appreciate that any suitable applicator may be used. In accordance with a preferred embodiment, the applicator is available from Pfeiffer of America, nasal gel pump 61476; address: 12 Roszel Road, Suite C-104, Princeton, N.J. 08540. This applicator is configured to hold about 50 metered 0.125 ml doses, of the composition.

The composition may be delivered to the patient in any suitable dosage. In accordance with one embodiment of the invention, the spray applicator is configured to supply a unit dose of about 0.125 ml of composition to the patient each time a pump associated with the spray applicator is activated (0.125 mls/spray). Preferably, the composition is delivered by applying 2-3 sprays to each nostril, for a total dosage of 4-6 sprays.

### EXAMPLES

The Examples set forth hereinbelow are illustrative of various aspects of certain preferred embodiments of the present invention. The compositions, methods and various parameters reflected therein are intended only to exemplify various aspects and embodiments of the invention, and are not intended to limit the scope of the claimed invention.

### Example 1

An exemplary gel composition for relieving migraine symptoms is prepared by admixing the following ingredients.

| | | Amount % | Homeopathic |
|---|---|---|---|
| Component | Function | w/w | Concentration |
| Chrysanthemum | Active | 4 | 1× |
| Leucanthemum | Ingredient | | |
| Magnesia Sulphurica | Active | 1 | 3× |
| | Ingredient | | |
| Magnesia Muriatica | Active | 1 | 3× |
| | Ingredient | | |
| Riboflavinum | Active | .003 | 6× |
| | Ingredient | | |
| Benzyl Alcohol | Preservative | 0.20% | |
| Disodium EDTA | Stabilizer | 0.10% | |
| Glycerin | Suspension | 1.00% | |
| | Agent | | |
| Hydroxyethyl cellulose | Thickener | 1.25% | |
| Potassium Chloride | pH Buffer | 0.2% | |
| Potassium Phosphate | pH Buffer | 0.012% | |
| Sodium Chloride | pH Buffer | 0.9 | |
| Sodium Phosphate | pH Buffer | 0.11 | |
| Benzalkonium Chloride | Preservative | 0.02 | |
| Solution | | | |
| Purified Water | Diluent | 90% | |

In accordance with one embodiment of the invention, a composition is formed by adding the water to a mixing vessel and starting a mixer to create a vortex. The sodium chloride is added to the water and mixed until the sodium chloride is dissolved. The Magnesia Sulphurica and the Magnesia Muriatica are then added and dissolved in the water mixture. Next, the potassium chloride, potassium phosphate and sodium phosphate are mixed in for about 5 minutes—until they are dissolved. The glycerin is then added to the solution, while continuously mixing. The disodium EDTA is then added and mixed for about 5 minutes until it dissolves. The benzyl alcohol and the benzalkonium chloride solution are then added and mixed for about five minutes. Dilute nasal spray mother solution of Chrysanthemum Leucanthemum (45% v/v whole plant in alcohol) and Riboflavinum are then added to the mixing vessel, while continuously stirring. Mixing is continued for about another 5 minutes. The hydroxyethyl cellulose is the added to the vessel . The hydroxyethyl cellulose is preferably added to a shoulder of the vortex in a controlled and consistent manner. Once all of the hydroxyethyl cellulose is added, the mixer is turned off. The consistency of the solution is checked by placing a sample in a Petri dish and visually inspecting the solution—looking for any lumps or inconsistency. If no lumps are visible and the solution appears consistent, the gel is in final form. If, on the other hand, lumps or inconsistency exists, the mixing is continued until neither exist.

The finished product exhibits the following characteristics:

Viscosity: 5000-9000 cps at 25° C. at 3 rpm with spindle T-A

pH: 6.50-8.0

Odor: slight riboflavin

Taste: N/A

Color: Dark Orange

Clarity: Slightly Cloudy

Microorganisms: Mold and Yeast less than 200 CFU/ml

The viscosity of the composition is believed to briefly fluctuate over time, but soon stabilizes at about three (3) months after initial preparation of the gel composition. For example, viscosity of the above formulation may be as follows:

t(0)=7,000 to 8,000 centipoise;

t(1 month)=4,000 to 5,000 centipoise;

t(2 months)=5,000 to 6,000 centipoise;

t(3 months)=4,500 to 5, 000 centipoise;

where t=the time from initial composition preparation, and viscosity is generally stabilized at three (3) months and greater.

### Example 2

An exemplary gel composition for relieving migraine symptoms is prepared by admixing the following ingredients in the manner described above.

| | | Amount % | Homeopathic |
|---|---|---|---|
| Component | Function | w/w | Concentration |
| Chrysanthemum | Active | 4 | 1× |
| Leucanthemum | Ingredient | | |
| Magnesia Sulphurica | Active | 2 | 1.5× |
| | Ingredient | | |
| Riboflavinum | Active | .003 | 6× |
| | Ingredient | | |
| Benzyl Alcohol | Preservative | 0.20% | |
| Disodium EDTA | Stabilizer | 0.10% | |
| Glycerin | Suspension | 1.00% | |
| | Agent | | |
| Hydroxyethyl cellulose | Thickener | 1.25% | |
| Potassium Chloride | pH Buffer | 0.2% | |
| Potassium Phosphate | pH Buffer | 0.012% | |
| Sodium Chloride | pH Buffer | 0.9 | |
| Sodium Phosphate | pH Buffer | 0.11 | |
| Benzalkonium Chloride | Preservative | 0.02 | |
| Solution | | | |
| Purified Water | Diluent | 90% | |

### Example 3

An exemplary gel composition for relieving migraine symptoms is prepared by admixing the following ingredients in the manner described above in connection with Example 1.

| | | Amount % | Homeopathic |
|---|---|---|---|
| Component | Function | w/w | Concentration |
| Chrysanthemum | Active | 4 | 1× |
| Leucanthemum | Ingredient | | |
| Magnesia Muriatica | Active | 2 | 1.5× |
| | Ingredient | | |
| Riboflavinum | Active | .003 | 6× |
| | Ingredient | | |
| Benzyl Alcohol | Preservative | 0.20% | |
| Disodium EDTA | Stabilizer | 0.10% | |
| Glycerin | Suspension | 1.00% | |
| | Agent | | |
| Hydroxyethyl cellulose | Thickener | 1.25% | |
| Potassium Chloride | pH Buffer | 0.2% | |
| Potassium Phosphate | pH Buffer | 0.012% | |
| Sodium Chloride | pH Buffer | 0.9 | |
| Sodium Phosphate | pH Buffer | 0.11 | |
| Benzalkonium Chloride | Preservative | 0.02 | |
| Solution | | | |
| Purified Water | Diluent | 90% | |

### Example 4

The delivery composition is delivered into the patient's nose using a nasal spray applicator. The patient is thereafter to prime the pump associated with the applicator prior to initial use by first removing a safety cap associated therewith, and then holding the bottle upright and pumping the pump applicator several times into a tissue until the pump applicator is primed. The pump applicator is primed where, for example, the gelled composition escapes out of the exterior of the applicator. Preferably, the pump applicator is configured as a metered-dose dispensing bottle configured to provide a specified amount of gelled composition to the patient.

Next, the patient is instructed to place the tip of the nasal pump just past the nasal opening, approximately ⅛ of an inch, to spray the delivery composition into the nostril by depressing the pump applicator, and to “sniff-up” the gel composition. The patient is instructed to deliver the composition into each nostril.

After application, the patient is instructed to depress the outside of each nostril for about five (5) seconds. The patient is thereafter instructed to reapply the composition to each nostril every two to four (2-4) hours until symptoms subside.

The present invention has been described above with reference to a number of exemplary embodiments and examples. It should be appreciated that the particular embodiments shown and described herein are illustrative of the invention and its best mode and are not intended to limit in any way the scope of the invention as set forth in the claims. For example, although the composition, system, and method have been described in detail in connection with application to a nasal membrane using a spray applicator, the composition may alternatively be applied to an oral membrane and may alternatively be applied to a nasal or oral membrane using other means such as swabs or the like. Those skilled in the art having read this disclosure will recognize that changes and modifications may be made to the exemplary embodiments without departing from the scope of the present invention. Further, though reference is made to “substances”, “agents”, and “ingredients”, skilled artisans will appreciate that these terms can be used interchangeably. Additionally, although reference is made separately to “gelled matrix”, “delivery composition”, and “carrier”, artisans will understand that these terms may be utilized both individually and interchangeably without departing from the scope of the invention. Additionally, though various components of the carrier are described herein in terms of exemplary embodiments, such as, for example, thickeners, permeations enhancers, emulsifiers, buffers, and preservatives, any suitable carrier may be achieved through any number or combination of additives now known or hereafter devised. Accordingly, these and other changes or modifications are intended to be included to be within the scope of the present invention, as expressed in the following claims.

1. A composition for treating symptoms associated with migraines, the composition comprising:
a carrier comprising water and a thickening agent; and
an effective amount of an active ingredient, the active ingredient configured to reduce a severity of a migraine symptom.

2. The composition of claim 1, wherein the active ingredient is selected from the group consisting of homeopathic and allopathic agents.

3. The composition of claim 1, wherein the active ingredient is selected from the group consisting of chrysanthemum leucanthemum, magnesia sulphurica, magnesia muriatica, and riboflavinum.

4. The composition of claim 1, wherein a viscosity of the composition is greater than about 2,500 centipoise.

5. The composition of claim 4, wherein the active ingredient comprises an agent selected from the group consisting of an anti-spasmodic agent, a calming agent, a vessel tone agent, and a serotonin antagonist.

6. The composition of claim 1, wherein the thickener comprises hydroxyethyl cellulose.

7. The composition of claim 1, further comprising a permeation enhancer.

8. The composition of claim 7, where the permeation enhancer comprises at least one of the following: liposomes, sequestering agents, ascorbic acid, glycerol, chitosan, lysophosphotidylcholin, EDTA, and disodium EDTA.

9. The composition of claim 1 further comprising a preservative.

10. The composition of claim 9, wherein the preservative comprises at least one of the following: benzalkonium chloride and benzyl alcohol.

11. The composition of claim 1 further comprising a buffer.

12. The composition of claim 11, wherein the buffer comprises at least one of the following: sodium phosphate, potassium phosphate, sodium chloride, and potassium chloride.

13. The composition of claim 1, wherein the carrier comprises glycerin.

14. The composition of claim 1, wherein the composition comprises about 0.05 to about 5.0 wt % glycerin.

15. The composition of claim 1, wherein the composition comprises about 0.0000001 to about 10 wt % of an active substance; about 0.05 to about 5.0 wt % glycerin; about 0 to about 5.0 wt % of a permeation enhancer; and about 0.0001 to about 1.0 wt % of a preservative.

16. A method of forming a migraine relief formula, the method comprising the steps of:
admixing fluid and buffering agents and dissolving the buffering agents in the fluid;
admixing active ingredients to the fluid and dissolved buffering agents; and
admixing a thickener to the fluid.

17. The method of claim 16, further comprising the step of mixing the fluid and the thickener until the mixture is substantially consistent and substantially lump-free.

18. A method of relieving symptoms associated with a migraine, the method comprising the steps of:
providing a migraine-relief formula comprising an active ingredient and a carrier; and
applying the formula to a nasal membrane.

19. A method of relieving symptoms associated with a migraine, the method comprising the steps of:
providing a migraine-relief formula having a viscosity greater than about 2,500 centipoise; and
applying the formula to an oral membrane.
